(19) United States
(12) Patent Application Publication
(10) Pub. No.: US 2024/0325182 A1
(43) Pub. Date: Oct. 3, 2024

(54) DEVICES, SYSTEMS, AND METHODS FOR CONTROLLING A POSITION OF AN IMPLANTABLE DEVICE

(71) Applicant: BOSTON SCIENTIFIC SCIMED, INC., Maple Grove, MN (US)

(72) Inventors: Mark Phillip Olson, New Brighton, MN (US); Raymond David Gessler, III, Roberts, WI (US); Nathan Stenger, St. Paul, MN (US); Matthew Maciej, Rogers, MN (US)

(73) Assignee: BOSTON SCIENTIFIC SCIMED, INC., Maple Grove, MN (US)

(21) Appl. No.: 18/622,297

(22) Filed: Mar. 29, 2024

Related U.S. Application Data
(60) Provisional application No. 63/455,842, filed on Mar. 30, 2023.

(51) Int. Cl. A61F 2/966; A61F 2/95; A61F 5/00

(52) U.S. Cl. CPC A61F 2/966; A61F 5/0036; A61F 5/0076; A61F 2002/9511

(57) ABSTRACT

A stent delivery system includes an outer shaft and an inner shaft extending at least partially within a lumen of the outer shaft. The inner shaft includes a stent receiving region, and a distal tip coupled to a distal end of the inner shaft. The distal tip includes a plurality of hooks positioned adjacent a proximal edge of the distal tip. A stent is disposed along the stent receiving region, and includes a first end region, a second end region, and one or more tethers coupled to the first end region of the stent. The one or more tethers are configured to engage with the plurality of hooks, thereby coupling the stent and the distal tip. The one of the one or more tethers may be configured to be actuated by a user to move the stent between a first, pre-deployed configuration and a second, deployed configuration.

### CROSS-REFERENCE TO RELATED APPLICATIONS

This application claims the benefit of U.S. Provisional Patent Application Ser. No. 63/455,842 filed on Mar. 30, 2023, the disclosure of which is incorporated herein by reference.

### TECHNICAL FIELD

The present disclosure relates generally to the field of implantable medical devices for adjusting accessibility through a passage of a medical device and related systems and methods. More particularly, the present disclosure relates to devices, systems, and methods for controlling a position of an implantable device, such as a lumen-apposing device, for regulating flow through a passage between two non-adherent structures.

### BACKGROUND

Treatment methods for various medical conditions, such as obesity, diabetes, or duodenal ulcers, involve bypassing the duodenum or restricting flow of materials through the duodenum. If the treatment requires complete bypass of the duodenum, then occlusion (e.g., full occlusion) of the pylorus may be indicated, and an anastomosis may be created, such as between the stomach and the jejunum. A lumen-apposing device may be placed between the stomach and the jejunum to allow for passage of materials (fluid, liquid, chyme, etc.) from the stomach and into the jejunum. One challenge presented by such devices is to prevent migration of the device. Thus, there is an ongoing need to provide alternative medical devices as well as alternative methods for manufacturing and using medical devices.

The present disclosure relates to devices, systems, and methods for controlling a position of an implantable device, such as a lumen-apposing device, for regulating flow through a passage between two non-adherent structures. Even more particularly, the present disclosure relates to devices, systems, and methods for controlling a position of an implantable device to prevent migration of the device.

### BRIEF SUMMARY

This summary of the disclosure is given to aid understanding, and one of skill in the art will understand that each of the various aspects and features of the disclosure may advantageously be used separately in some instances, or in combination with other aspects and features of the disclosure in other instances. No limitation as to the scope of the claimed subject matter is intended by either the inclusion or non-inclusion of elements, components, or the like in this summary. Accordingly, while the disclosure is presented in terms of aspects or embodiments, it should be appreciated that individual aspects can be claimed separately or in combination with aspects and features of that embodiment or any other embodiment.

An example medical device may include a stent delivery system. The stent delivery system may include an outer shaft having a distal end region and an inner surface defining a lumen extending therein, an inner shaft extending at least partially within the lumen of the outer shaft, the inner shaft having a stent receiving region disposed along a distal end region thereof, and a distal tip coupled to a distal end of the distal end region of the inner shaft, the distal tip having a plurality of hooks positioned adjacent a proximal edge of the distal tip. A stent may be disposed along the stent receiving region, the stent having a first end region, a second end region, and a medial region positioned between the first end region and the second end region, and one or more tethers coupled to the first end region of the stent, the one or more tethers configured to extend from the first end region to the distal tip and configured to engage with the plurality of hooks, thereby coupling the stent and the distal tip. One of the one or more tethers may extend proximally through the lumen along an outer surface of the inner shaft, wherein the one of the one or more tethers may be configured to be actuated by a user, and the stent may be configured to move between a first, pre-deployed configuration and a second, deployed configuration, the stent being held in the first, pre-deployed configuration when positioned within the lumen of the outer shaft.

Alternatively or additionally to any of the embodiments above, the distal tip may be a cauterization tip.

Alternatively or additionally to any of the embodiments above, the first end region may include a first flange structure and the second end region may include a second flange structure.

Alternatively or additionally to any of the embodiments above, the one or more tethers may include a singular tether configured to be threaded within the first end region of the stent and form a plurality of loops configured to engage with the plurality of hooks.

Alternatively or additionally to any of the embodiments above, the singular tether may include a first end and a second end, the second end configured to extend proximally through the lumen along the outer surface of the inner shaft.

Alternatively or additionally to any of the embodiments above, the one of the one or more tethers may be coupled to a ratchet mechanism, wherein actuation of the ratchet mechanism causes the distal end region of the stent to expand.

Alternatively or additionally to any of the embodiments above, the first end region of the stent may include a plurality of hooks configured to engage with the one or more tethers.

Alternatively or additionally to any of the embodiments above, proximal retraction of the outer shaft may uncover at least a portion of the stent, and wherein the uncovered portion of the stent expands radially outward as the outer shaft is proximally retracted.

Alternatively or additionally to any of the embodiments above, when the outer shaft has been fully retracted, the stent may expand to the second, deployed position, wherein the first end region is positioned adjacent a first tissue structure and the second end region may be positioned adjacent a second tissue structure.

Alternatively or additionally to any of the embodiments above, when the stent is in the second, deployed position, the inner shaft may be proximally retracted and the one or more tethers disengage from the first end region of the stent and retract proximally with the inner shaft.

Another example medical device may include a stent delivery system. The stent delivery system may include an outer shaft having a distal end region and an inner surface defining a lumen extending therein, an inner shaft extending at least partially within the lumen of the outer shaft, the inner shaft having a stent receiving region disposed along a distal end region thereof, a distal tip coupled to a distal end of the distal end region of the inner shaft, the distal tip having a plurality of hooks positioned adjacent a proximal edge of the distal tip, a stent disposed along the stent receiving region, the stent having a first end region, a second end region, and a medial region positioned between the first end region and the second end region, and one or more tethers coupled to the first end region of the stent, the one or more tethers configured to extend from the first end region to the distal tip and configured to engage with the plurality of hooks, thereby coupling the stent and the distal tip.

Alternatively or additionally to any of the embodiments above, the outer shaft may be retracted proximally relative to the inner shaft to expo se the first end region of the stent and permit the first end region of the stent to move from a first, pre-deployed position to a second, deployed position.

Alternatively or additionally to any of the embodiments above, one of the one or more tethers may extend proximally through the lumen along an outer surface of the inner shaft, wherein the one of the one or more tethers may be configured to be actuated by a user.

Alternatively or additionally to any of the embodiments above, actuation of the one of the one or more tethers may exert a proximal force on the distal end region of the stent, causing the first end region to expand radially outward.

Alternatively or additionally to any of the embodiments above, when the stent is in the second, deployed position, the inner shaft is proximally retracted and the one or more tethers disengage from the first end region of the stent and retract proximally with the inner shaft.

Another example may include a method of treating a body lumen. The method may include advancing a stent delivery system to a target tissue site, the stent delivery system may include an outer shaft having a distal end region and an inner surface defining a lumen extending therein, an inner shaft extending at least partially within the lumen of the outer shaft, the inner shaft having a stent receiving region disposed along a distal end region thereof, a distal tip coupled to a distal end of the distal end region of the inner shaft, the distal tip having a plurality of hooks positioned adjacent a proximal edge of the distal tip, a stent disposed along the stent receiving region, the stent having a first end region, a second end region, and a medial region positioned between the first end region and the second end region, and one or more tethers coupled to the first end region of the stent, the one or more tethers configured to extend from the first end region to the distal tip and configured to engage with the plurality of hooks, thereby coupling the stent and the distal tip. The method may include retracting the outer shaft proximally relative to the inner shaft to expose the first end region of the stent and permit the first end region of the stent to move from a first, pre-deployed position to a second, deployed position, actuating the one or more tethers in a proximal direction, thereby applying a proximal force to the first end region of the stent causing the first end region to expand radially outward, further retracting the outer shaft proximally relative to the inner shaft to expose the second end region of the stent and permit the second end region of the stent to move from the first, pre-deployed position to the second, deployed position, and retracting the inner shaft proximally thereby causing the one or more tethers to disengage from the first end region of the stent and retract proximally with the inner shaft.

Alternatively or additionally to any of the embodiments above, while applying the proximal force to the first end region, positioning the first end region of the stent adjacent a first tissue structure such that the first tissue structure is moved in a proximal direction and engages with a second tissue structure.

Alternatively or additionally to any of the embodiments above, the one or more tethers includes a singular tether configured to be threaded within the first end region of the stent and form a plurality of loops configured to engage with the plurality of hooks.

Alternatively or additionally to any of the embodiments above, the first end region includes a first flange structure and the second end region includes a second flange structure, wherein the proximal force applied to the first end region causes the first flange structure to include an outer diameter greater than an outer diameter of the second flange structure.

Alternatively or additionally to any of the embodiments above, one of the one or more tethers extends proximally through the lumen along an outer surface of the inner shaft, wherein the one of the one or more tethers is configured to be actuated by a user.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 depicts a schematic plan view of an illustrative catheter;

FIG. 2 depicts a schematic partial view of an elongated shaft of an illustrative catheter, illustrating an outer shaft, an inner shaft, and a distal tip;

FIG. 3 depicts a schematic enlarged view of a distal end region of an inner shaft, as in Circle 3 of FIG. 2;

FIG. 4 depicts a schematic illustrative catheter extending through a tissue wall;

FIG. 5 depicts a schematic illustrative catheter partially extending through a tissue wall and a partial deployment of an illustrative stent;

FIG. 6 depicts a schematic illustrative stent positioned between two adjacent tissue walls;

FIG. 7 depicts a schematic side view of a stent positioned between a stomach and a portion of a small intestine;

FIG. 8 depicts a schematic cross-sectional view the stent positioned between the stomach and the portion of the small intestine taken at line 8-8 of FIG. 7;

FIG. 9 depicts a schematic side view of a stent positioned over an inner shaft coupled to a distal tip; and

FIG. 10 depicts a schematic perspective view of the stent of FIG. 9 positioned over the inner shaft with a first end region of the stent in a deployed position.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

In accordance with various principles of the present disclosure, an implantable device may be used to extend across an anatomical structure to control or regulate the size of a passage therethrough. For instance, an implantable device may extend across a body passage or lumen, such terms being used interchangeably herein without intent to limit. The body passage or lumen may include, without limitation, a portion of a passage or lumen, a passage or lumen between anatomical structures (passages, lumens, cavities, organs, etc.), a passage created across apposed tissue walls (such as to create an anastomosis) etc. The device has a passage or lumen (such terms being used interchangeably herein without intent to limit) therethrough which may be used to occlude or block or narrow or close or constrict or regulate or control (such terms and conjugations thereof may be used interchangeably herein without intent to limit) the body passage through which the device is positioned. The device may be considered and referenced as an occlusion or lumen-apposing or anastomosis or flow-regulating or flow-controlling device, and such terms and various other alternatives thereto may be used interchangeably herein without intent to limit.

It will be appreciated that devices, systems, and methods as disclosed herein may be used in endoscopic, laparoscopic, and/or open surgical procedure. Preferably, a medical professional may be able to deliver and/or to remove the device endoscopically.

Advantageously, devices and systems disclosed herein may be used in minimally invasive procedures such as natural orifice transluminal endoscopic surgery.

The following detailed description should be read with reference to the drawings in which similar elements in different drawings are numbered the same. The drawings, which are not necessarily to scale, depict illustrative embodiments and are not intended to limit the scope of the claims.

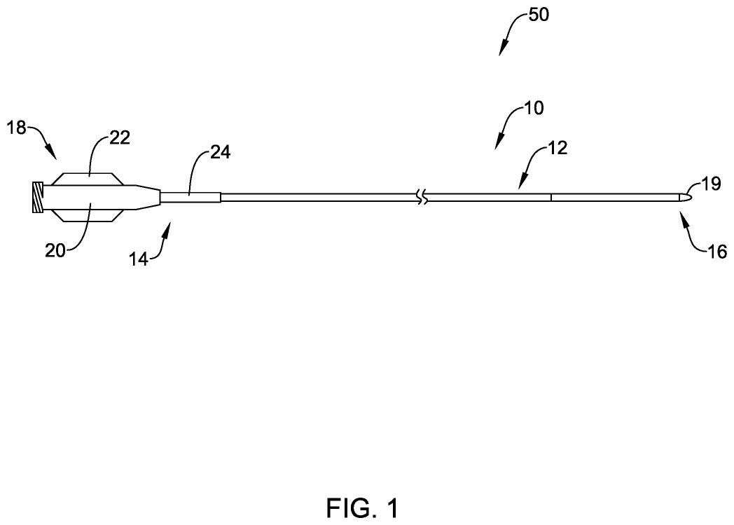

FIG. 1 depicts a schematic plan view of an illustrative stent delivery system 50 including a catheter 10. As shown in FIG. 1, the catheter 10 may include an elongated shaft 12 having a distal end 16 and a proximal end 14. The catheter 10 may have a length that is in the range of about 50 to 200 centimeters and may have an outer diameter (OD) that is less than 3 French. In some cases, the catheter 10 may have an OD of 1.7 French, 2 French, 2.5 French, 2.8 French, or any other suitable outer diameter. In some cases, the catheter 10 may include an OD of about 2.6 French at the distal end 16, and an OD of about 2.8 French at the proximal end 14. In some cases, the catheter 10 may have an inner diameter (ID) of about 0.025 inches to 0.027 inches. In other cases, the catheter 10 may have an ID of about 0.02 inches, 0.03 inches, or any other suitable inner diameter. These sizes may vary depending on particular usage.

In some cases, a distal tip 19 may be connected to or disposed about the distal end 16 of the elongated shaft 12. In some cases, the distal tip 19 may be a polymeric distal tip, which may be formed from an elastomer (e.g., Pebax®), a thermoplastic polymer, or any other suitable polymer. The distal tip 19 may be formed of a softer material than other portions of the outer shaft of the catheter 10, such as by using a polymer or elastomer having a shore hardness of less than 40D. In some cases, the distal tip 19 may be formed from a polymer with a shore hardness of about 35D. In some cases, the distal tip 19 may be a cauterization tip configured to create an opening in a tissue wall. These are just examples.

In some cases, as shown, a hub and strain relief assembly 18 may be connected to or disposed about the proximal end 14 of the elongated shaft 12. The hub and strain relief assembly 18 may include a main body portion 20, a pair of flanges 22 designed to improve gripping, and a strain relief 24 intended to reduce kinking The hub and strain relief assembly 18 may be a conventional design and attached by conventional techniques.

In some cases, the catheter 10 may be considered as being an over the wire (OTW) microcatheter, configured for passing the entire length thereof over a guidewire. It will be appreciated that in cases in which the catheter 10 is instead a rapid exchange catheter, a side port may be provided to allow the guidewire to exit the catheter lumen distal of the proximal hub and strain relief assembly 18. These are just examples.

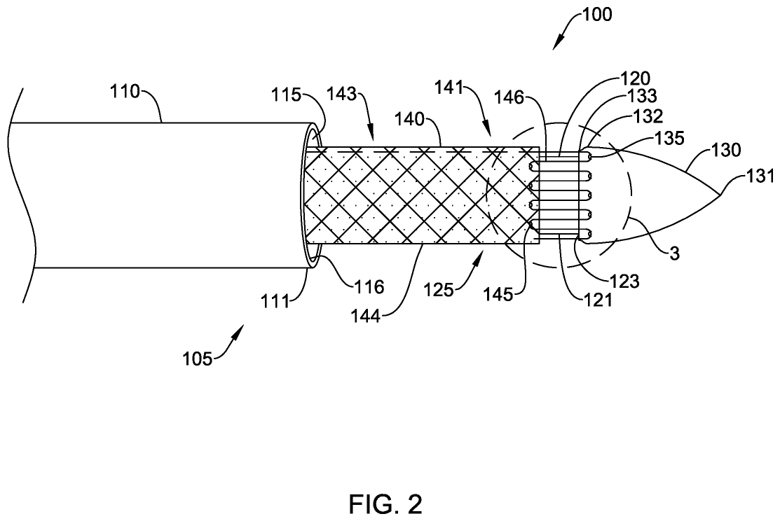

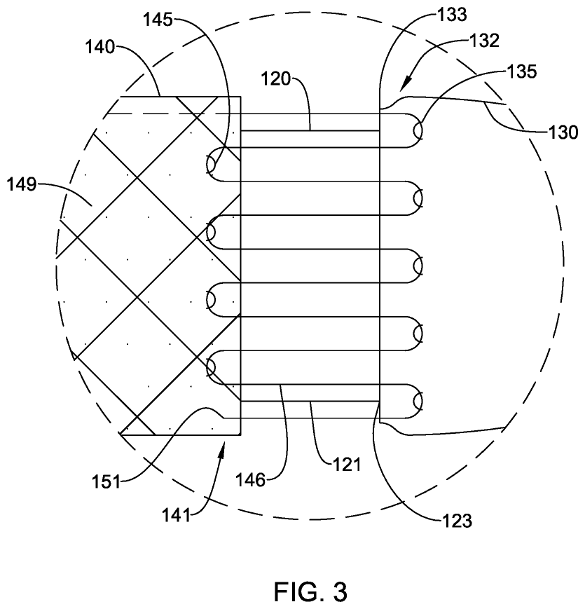

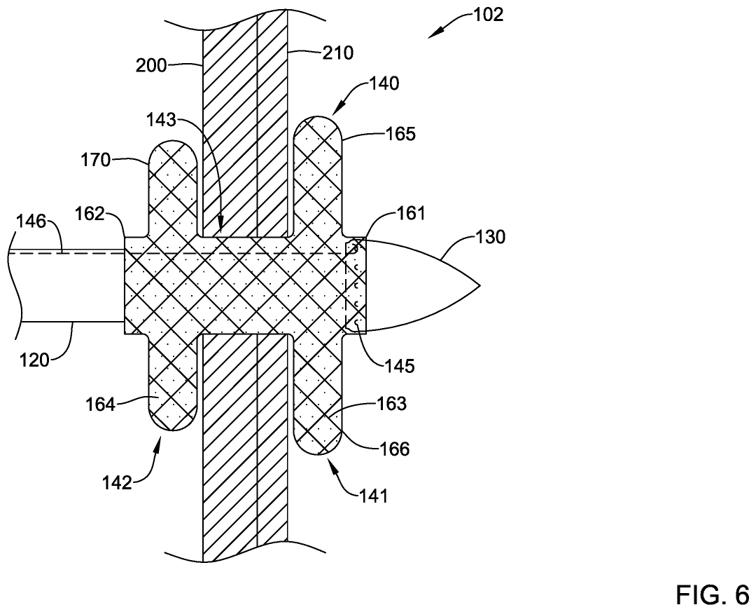

FIG. 2 depicts a schematic partial view of an elongated shaft 112 of an illustrative catheter 100, illustrating an outer shaft 110, an inner shaft 120, and a distal tip 130, and FIG. 3 depicts a schematic enlarged view of a distal end region 121 of the inner shaft 120, as in Circle 3 of FIG. 2. The catheter 100 may be considered to be an example of the catheter 10 of FIG. 1. The elongated shaft 112 may include the outer shaft 110 having a distal end region 111 and an inner surface 116 defining a lumen 115 extending therethrough. The inner shaft 120 may extend at least partially within the lumen 115 of the outer shaft 110, and the inner shaft 120 may include a stent receiving portion 125 disposed along a distal end region 121 thereof. A stent 140 may be disposed along the stent receiving portion 125. The stent 140 may include a first end region, a second end region, and a medial region positioned between the first end region and the second end region, as shown in FIGS. 6 to 10.

The catheter 100 may include the distal tip 130. The distal tip 130 may include a distal end 131 and a proximal end 132. The proximal end 132 of the distal tip 130 may be coupled to a distal end 123 of the distal end region 121 of the inner shaft 120. In some cases, the distal tip 130 may include a plurality of hooks 135 positioned adjacent a proximal edge 133 of the proximal end 132 of the distal tip 130. The plurality of hooks 135 may be configured to be positioned equidistantly around an outer diameter of the proximal end 132 of the distal tip 130. In some cases, the plurality of hooks 135 may include a range of about 2 to 15 hooks. In some cases, the plurality of hooks 135 may include more than 15 hooks, or any number of hooks as desired.

In some cases, one or more tethers 146 may be coupled to a first end region 141 of the stent 140. The one or more tethers 146 may be configured to extend from the first end region 141 of the stent 140 to the distal tip 130 and may be configured to engage with the plurality of hooks 135 at the proximal end 132 of the distal tip 130, thereby coupling the stent 140 to the distal tip 130. In some cases, the one or more tethers 146 may include a singular tether configured to be threaded within the first end region 141 of the stent 140, such as, for example, within interstices 166 of a radially expanding tubular framework 144. When the one or more tethers 146 are threaded within the first end region 141 of the stent 140, the one or more tethers 146 may form a plurality of loops configured to engage with the plurality of hooks 135 of the distal tip 130. In some cases, the first end region 141 of the stent 140 may include a plurality of hooks 145, and the one or more tethers 146 may form a plurality of loops and may be configured to engage with the plurality of hooks 145 and the plurality of hooks 135. In some cases, when the one or more tethers 146 includes a singular tether, the tether may include a first end 151 and a second end (not explicitly shown in FIGS. 2 to 3). The second end may be configured to extend proximally through the lumen 115 of the outer shaft 110 along an outer surface of the inner shaft 120 and to a proximal end of the catheter 100. In some cases, the one or more tethers 146 may be formed from synthetic polymer materials, such as polydioxanone, polyglycolic acid, polyglyconate, and polylactic acid, nylon, silk, or the like.

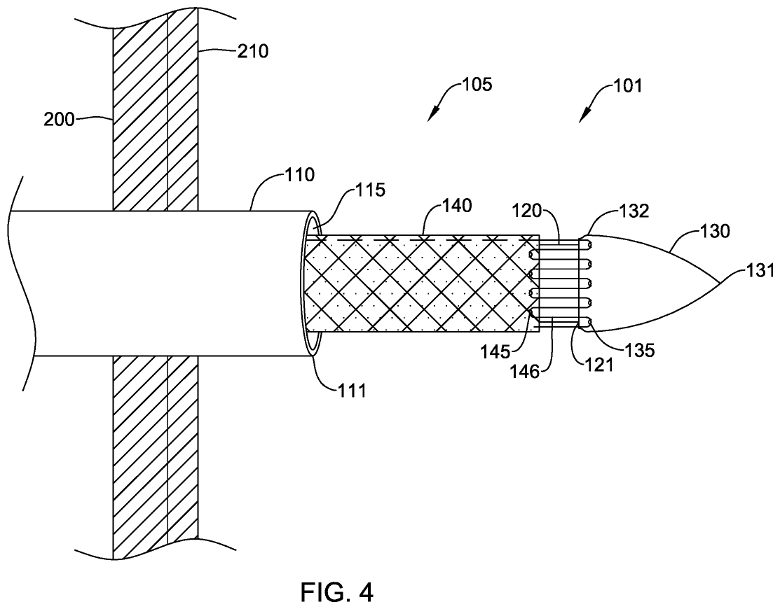

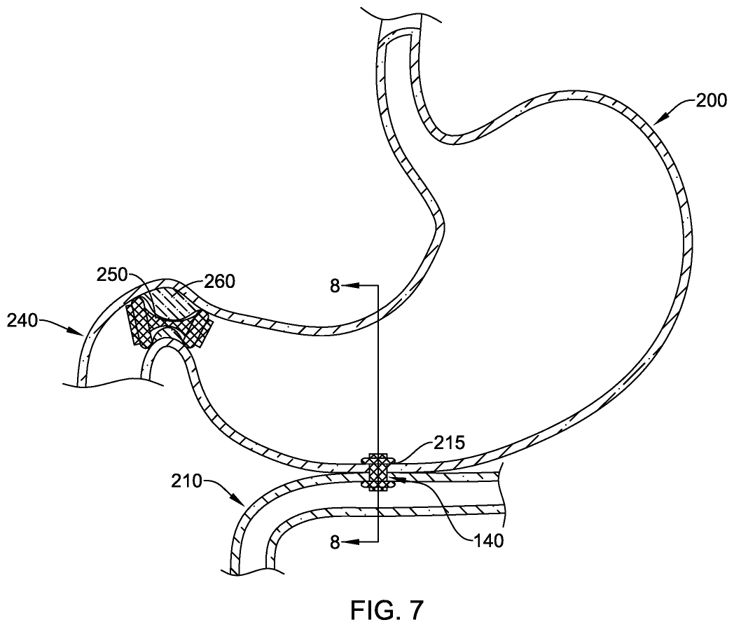

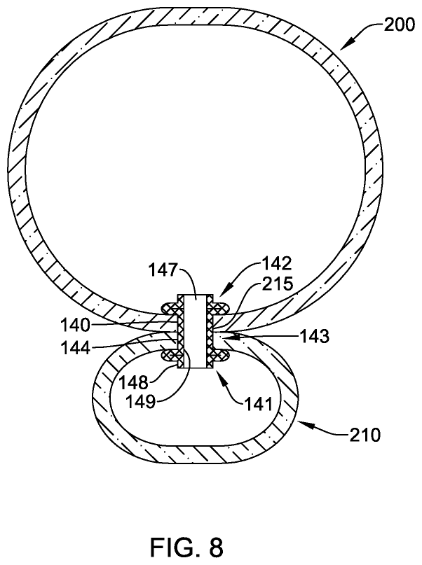

FIGS. 4 to 6 depict an illustrative method of deploying the stent 140 across two adjacent tissues, for example, a stomach 200 and a portion of a small intestine 210. FIG. 7 depicts a schematic side view of the stent 140 positioned between the stomach 200 and a portion of the small intestine 210, and FIG. 8 depicts a schematic cross-sectional view the stent 140 positioned between the stomach 200 and the portion of the small intestine 210 taken at line 8-8 of FIG. 7.

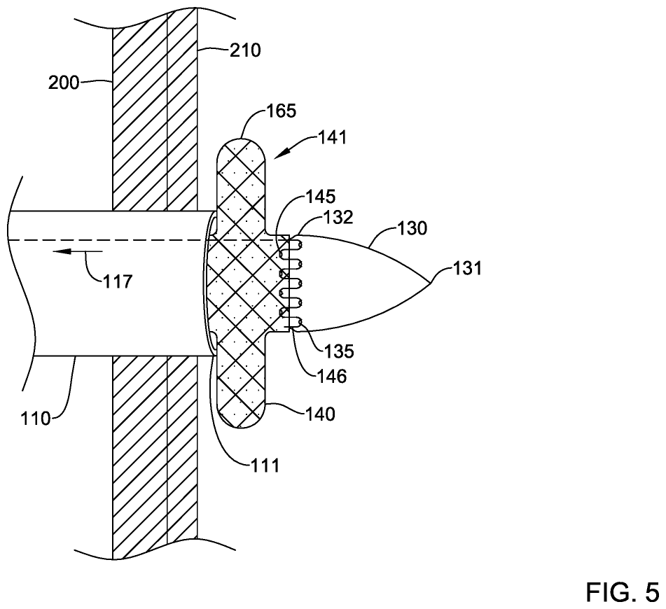

The method may include advancing a stent delivery system 50 to a target tissue site. As shown in FIG. 4, the catheter 100 has passed through the tissue walls of the stomach 200 and the portion of the small intestine 210. In the example shown, the distal tip 130, which may be a cauterization tip, was pressed against a tissue wall to cauterize the tissue walls (e.g., the stomach 200 and the portion of the small intestine 210). As the catheter 100 passes through the tissue walls, the outer shaft 110 abuts the distal tip 130, thereby fully covering the stent 140, and the outer shaft 110 holds the stent 140 in a first, pre-deployed configuration 101. The outer shaft 110 may be retracted proximally, thereby uncovering (e.g., exposing) at least a portion of the stent 140 which is disposed about the inner shaft 120. As the outer shaft 110 is retracted proximally, the uncovered portion of the stent 140 expands radially outward from the first, pre-deployed configuration 101 (FIG. 4) to a second, deployed configuration 102 (FIGS. 5 to 6). The one or more tethers 146 may be configured to be actuated in a proximal direction, as indicated by arrow 117 in FIG. 5, which may pull on the plurality of hooks 135 at the proximal end 132 of the distal tip 130, which then abuts the first end region 141 of the stent 140 to expand the first end region 141 of the stent 140 such that the first end region 141 includes a first flange structure 165, as shown in FIGS. 5 to 6. In some cases, the one or more tethers 146 may be actuated by a user, in which a user (e.g., a physician) may pull the one or more tethers proximally. In some cases, the one or more tethers 146 may be coupled to a ratchet mechanism, wherein actuation of the ratchet mechanism pulls the one or more tethers 146 proximally, thereby causing the first end region 141 of the stent 140 to expand.

In some cases, as the one or more tethers 146 are actuated proximally, a user may further retract the catheter 100 in a proximal direction. This may allow the user to direct the placement of the first flange structure 165 during deployment of the stent 140. For example, a user may retract the catheter 100 proximally and actuate (e.g., pull or ratchet) the one or more tethers 146 to position the first flange structure 165 along a distal side of a first tissue structure (e.g., the portion of the small intestine 210). Continual actuation of the one or more tethers 146 may place a pulling force on the distal tip 130, which then presses on the first end region 141 of the stent 140, expanding the first flange structure 165 to the desired size, which then pulls the portion of the small intestine 210 towards the stomach 200.

Once the first flange structure 165 is in the desired position, the outer shaft 110 may be fully retracted, and the second end region 142 of the stent 140 may expand to include a second flange structure 170. The second flange structure 170 may be positioned such that it abuts a distal side of a second tissue structure (e.g., the stomach 200), as shown in FIGS. 6 to 8. Upon placement of the stent 140, the inner shaft 120 including the distal tip 130 may be retracted proximally. When the inner shaft 120 is retracted, the one or more tethers 146 may be released, and may disengage from the first end region 141 of the stent 140 and may be removed (e.g., retracted) with the inner shaft 120 and the distal tip 130. In some cases, the one or more tethers 146 may be cut at any location along the second end 152 of the one or more tethers 146, which may allow the one or more tethers 146 to disengage from the first end region 141 of the stent 140 and be removed (e.g., retracted) with the inner shaft 120 and the distal tip 130.

The stent 140 may be a self-expanding stent 140 and may include the radially expanding tubular framework 144 having a radially outward surface 148 and a radially inward surface 149. The term ‘radially expanding tubular framework 144’ may be referred to as ‘tubular framework 144’ hereafter. In some cases, the stent 140 may include a height of 10 millimeters (mm) and an outer diameter (e.g., width) of 20 mm. In some cases, the height of the stent 140 may be 12 mm, 15 mm, 18 mm, between 12 mm and 18 mm, or any other suitable height. In some cases, the outer diameter of the stent 140 may be 18 mm, 22 mm, 25 mm, between 18 mm and 25 mm, or any other suitable diameter.

The tubular framework 144 may include the first end region 141, the second end region 142, and the medial region 143 positioned between the first end region 141 and the second end region 142. The tubular framework 144 may further include a lumen 147 extending from the first end region 141 to the second end region 142. In some cases, the first end region 141 may be considered to be a distal end region, and the second end region 142 may be considered to be a proximal end region. In alternative cases, the first end region 141 may be considered to be a proximal end region, and the second end region 142 may be considered to be a distal end region. The first end region 141 may extend from a first end 161 to the medial region 143, and the second end region 142 may extend from a second end 162 to the medial region 143. The medial region 143 may define a midpoint in the tubular framework 144, such that the first end region 141 and the second end region 142 may have the same lengths. Alternatively, the medial region 143 may be disposed at a location other than a midpoint, such that the first and second end regions 141, 142 have different lengths.

In some cases, the first end region 141 may include the first flange structure 165 and the second end region 142 may include the second flange structure 170. The medial region 143 may be positioned between the first flange structure 165 and the second flange structure 170. The first flange structure 165 and the second flange structure 170 may be considered to be retention members configured to aid in holding the stent 140 in place. Thus, the first and second flange structures 165, 170 may include a width (e.g., an outer diameter) sufficient to provide retention strength. For example, the width of the first and second flange structures 165, 170 may be in the range of 20 to 70 mm. In some cases, the first and second flange structures 165, 170 may include a width greater than that of the first end 161, the second end 162, and the medial region 143 of the tubular framework 144. In some cases, the first and second flange structures 165, 170 may include the same width. In some cases, the first and second flange structures 165, 170 may include differing widths. In some cases, the first and second flanges 165, 170 may include any of a variety of shapes, such as concave, convex, disc-shaped, cylindrical (e.g., having a longer longitudinal extent then illustrated), etc., or other configurations, the particular shape and configuration not being limited by the present disclosure. While it is illustrated that the first flange structure 165 is positioned near the first end region 141 and the second flange structure 170 is positioned near at the second end region 142, it may be contemplated that the first flange structure 165 is positioned near the second end region 142 and the second flange structure 170 is positioned near the first end region 141. In some cases, it may be contemplated that the tubular framework 144 includes only one flange structure (e.g., the first flange structure 165 or the second flange structure 170).

The stent 140 may be configured to be implanted between the stomach 200 and the jejunum 210 of a patient, to form an anastomosis 215. In other embodiments, the stent 140 may be configured to be implanted in the urinary, biliary, tracheobronchial, esophageal or renal tracts, for example. Since the stent 140, or a portion thereof, may be intended to be implanted permanently in the body lumen, the stent 140 may be made, at least in part, from a biostable material. Examples of the biostable metal materials may include, but are not limited to, stainless steel, tantalum, tungsten, niobium, platinum, nickel-chromium alloys, cobalt-chromium alloys such as Elgiloy® and Phynox®, nitinol (e.g., 55% nickel, 45% titanium), cisplatin, and other alloys based on titanium, including nickel titanium alloys, or other suitable metals, or combinations or alloys thereof. Some suitable biostable polymeric materials include, but are not necessarily limited to, polyimide, polyether block amide, polyethylene, polyethylene terephthalate, polypropylene, polyvinylchloride, polyurethane, polytetrafluoroethylene, polysulfone, and copolymers, blends, mixtures or combinations thereof.

The tubular framework 144 may include a number of interconnected struts 163 to form a mesh-like structure of the tubular framework 144. The struts 163 may be configured to transition from a compressed state to an expanded state. The struts 163 may include a diameter of, for example, 0.0762 mm to 0.3556 mm. The tubular framework 144 may include a coating 164 applied over the struts 163 of the tubular framework 144, thus the entirety of the stent 140 may be covered with the coating 164. The coating 164 may be formed from a silicone and may be configured to prevent leakage of food materials during anastomosis formation. In some cases, the coating 164 may be applied over the struts 163 in the medial region 143. In some cases, the coating 164 may be applied over the struts 163 within the first end region 141 and the medial region 143, and in some cases, the coating 164 may be applied over the struts 163 within the second end region 142 and the medial region 143. These are just examples.

The stomach 200 normally passes food materials (e.g., chyme, partially digested food materials, fluids, etc.) into a duodenum 240 through a pylorus 260. In some cases, treatment for a patient experiencing obesity, diabetes, or duodenal ulcers, may involve bypassing the duodenum 240, or restricting flow of materials through the duodenum 240. If the treatment requires complete bypass of the duodenum 240, then occlusion (e.g., full occlusion) of the pylorus 260 may be indicated, and an anastomosis 215 may be created between the stomach 200 and the jejunum 210, which may be known as a gastrojejunostomy. FIG. 7 illustrates an example bypass procedure in which a flow restricting device 250 has been positioned within the pylorus 260, thereby restricting access of food materials from the stomach 200 into the duodenum 240 (e.g., a complete bypass). A lumen-apposing metal stent (LAMS), such as the stent 140, may be placed between the stomach 200 and the jejunum 210, thereby forming the anastomosis 215, to allow for passage of food materials (fluid, liquid, chyme, etc.) from the stomach 200 and into the jejunum 210, as shown in FIGS. 7 to 8. While it is illustrated that the stent 140 may be used in forming the anastomosis 215 between the stomach 200 and the jejunum 210, it may be contemplated that the stent 140 may be used to treat a stenosis in a blood vessel, used to maintain a fluid opening or pathway in the vascular, urinary, biliary, tracheobronchial, esophageal or renal tracts, or position a device such as an artificial valve or filter within a body lumen, in some instances. Although illustrated as a stent, the stent 140 may be any of a number of devices that may be introduced endoscopically, subcutaneously, percutaneously or surgically to be positioned within an organ, tissue, or lumen, such as a heart, artery, vein, urethra, esophagus, trachea, bronchus, bile duct, or the like.

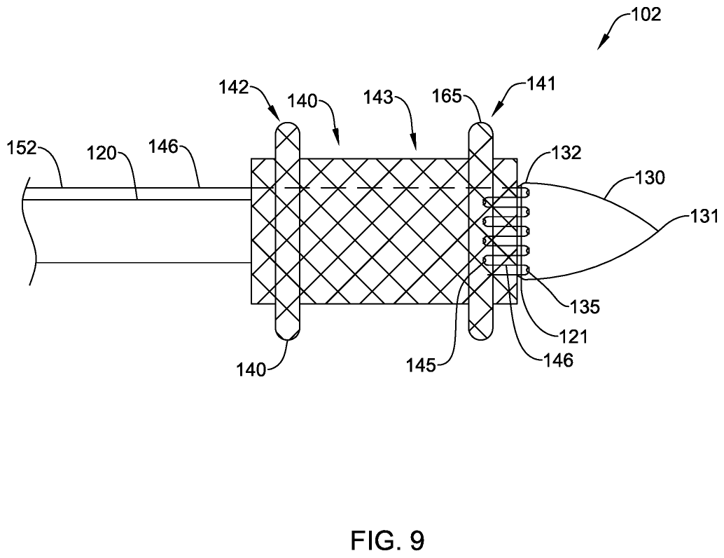

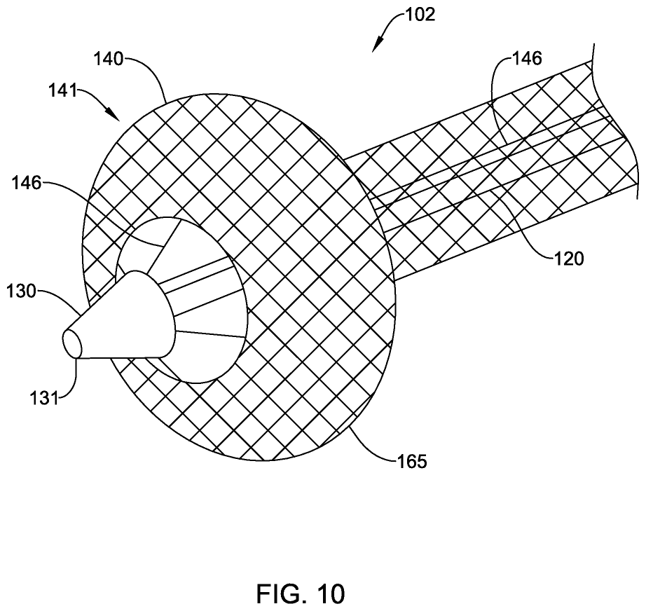

FIG. 9 depicts a schematic side view of the stent 140 positioned over the inner shaft 120 coupled to the distal tip 130, and FIG. 10 depicts a schematic perspective view of the stent 140 positioned over the inner shaft 120 with the first end region 141 of the stent in the deployed configuration 102. As discussed, with reference to FIGS. 4 to 8, the outer shaft 110 (not shown in FIGS. 9 to 10) may be retracted proximally, thereby uncovering (e.g., exposing) at least a portion of the stent 140 which is disposed about the inner shaft 120. As the outer shaft 110 is retracted proximally, the uncovered portion of the stent 140 expands radially outward from the first, pre-deployed configuration 101 (FIG. 4) to a second, deployed configuration 102 (FIGS. 9 to 10). The one or more tethers 146 may be configured to be actuated in a proximal direction, which may pull on the plurality of hooks 135 at the proximal end 132 of the distal tip 130, which then abuts the first end region 141 of the stent 140 to expand the first end region 141 of the stent 140 such that the first end region 141 includes the first flange structure 165 and the second end region 142 of the stent 140 may expand to include the second flange structure 170.

In some cases, as the one or more tethers 146 are actuated proximally which may place a pulling force on the distal tip 130, which then exerts a proximal force on the first end region 141 of the stent 140, causing the first flange structure 165 to expand radially outward to a desired size, which then pulls a first tissue structure towards a second tissue structure. While it has been discussed that the first and second tissue structures may include the stomach and a portion of the small intestine, it may be contemplated that the first and second tissue structures may be any two adjacent, non-adherent structures, such as in a hepaticogastrotomy, a gall bladder drainage, or the like.

In some cases, actuating (e.g., pulling) the one or more tethers 146 may allow the user to manipulate the size of the first flange structure 165 as needed to pull a distal tissue structure towards a proximal tissue structure. In use, the distal tip 130 is actuated (e.g., pulled) proximally, which can be pulled or ratcheted to a specified tension, causing the first flange structure 165 to expand and become rigid, as shown in FIG. 10. This dilates an outer diameter of the first flange structure 165 ensuring a proper anchor to a distal tissue structure.

The stent 140, and parts thereof, may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyimide (for example, DURETHAN® available from Bayer or CRISTAMID® available from Elf Atochem), elastomeric polyamides, block polyimide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In at least some embodiments, portions or all of stent 140 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of stent 140 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of stent 140 to achieve the same result.

In some embodiments, a degree of Magnetic Resonance Imaging (MRI) compatibility is imparted into stent 140. For example, stent 140 or portions thereof, may be made of a material that does not substantially distort the image and create substantial artifacts (i.e., gaps in the image). Certain ferromagnetic materials, for example, may not be suitable because they may create artifacts in an MRI image. The stent 140, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A stent delivery system, comprising:
an outer shaft having a distal end region and an inner surface defining a lumen extending therein;
an inner shaft extending at least partially within the lumen of the outer shaft, the inner shaft having a stent receiving region disposed along a distal end region thereof;
a distal tip coupled to a distal end of the distal end region of the inner shaft, the distal tip having a plurality of hooks positioned adjacent a proximal edge of the distal tip;
a stent disposed along the stent receiving region, the stent having a first end region, a second end region, and a medial region positioned between the first end region and the second end region; and
one or more tethers coupled to the first end region of the stent, the one or more tethers configured to extend from the first end region to the distal tip and configured to engage with the plurality of hooks, thereby coupling the stent and the distal tip;
wherein one of the one or more tethers extends proximally through the lumen along an outer surface of the inner shaft, wherein the one of the one or more tethers is configured to be actuated by a user; and
wherein the stent is configured to move between a first, pre-deployed configuration and a second, deployed configuration, the stent being held in the first, pre-deployed configuration when positioned within the lumen of the outer shaft.

2. The stent delivery system of claim 1, wherein the distal tip is a cauterization tip.

3. The stent delivery system of claim 1, wherein the first end region includes a first flange structure and the second end region includes a second flange structure.

4. The stent delivery system of claim 1, wherein the one or more tethers includes a singular tether configured to be threaded within the first end region of the stent and form a plurality of loops configured to engage with the plurality of hooks.

5. The stent delivery system of claim 4, wherein the singular tether includes a first end and a second end, the second end configured to extend proximally through the lumen along the outer surface of the inner shaft.

6. The stent delivery system of claim 1, wherein the one of the one or more tethers is coupled to a ratchet mechanism, wherein actuation of the ratchet mechanism causes the distal end region of the stent to expand.

7. The stent delivery system of claim 1, wherein the first end region of the stent includes a plurality of hooks configured to engage with the one or more tethers.

8. The stent delivery system of claim 1, wherein proximal retraction of the outer shaft uncovers at least a portion of the stent, and wherein the uncovered portion of the stent expands radially outward as the outer shaft is proximally retracted.

9. The stent delivery system of claim 8, wherein when the outer shaft has been fully retracted, the stent expands to the second, deployed position, wherein the first end region is positioned adjacent a first tissue structure and the second end region is positioned adjacent a second tissue structure.

10. The stent delivery system of claim 9, wherein when the stent is in the second, deployed position, the inner shaft is proximally retracted and the one or more tethers disengage from the first end region of the stent and retract proximally with the inner shaft.

11. A stent delivery system, comprising:
an outer shaft having a distal end region and an inner surface defining a lumen extending therein;
an inner shaft extending at least partially within the lumen of the outer shaft, the inner shaft having a stent receiving region disposed along a distal end region thereof;
a distal tip coupled to a distal end of the distal end region of the inner shaft, the distal tip having a plurality of hooks positioned adjacent a proximal edge of the distal tip;
a stent disposed along the stent receiving region, the stent having a first end region, a second end region, and a medial region positioned between the first end region and the second end region; and
one or more tethers coupled to the first end region of the stent, the one or more tethers configured to extend from the first end region to the distal tip and configured to engage with the plurality of hooks, thereby coupling the stent and the distal tip.

12. The stent delivery system of claim 11, wherein the outer shaft is retracted proximally relative to the inner shaft to expose the first end region of the stent and permit the first end region of the stent to move from a first, pre-deployed position to a second, deployed position.

13. The stent delivery system of claim 11, wherein one of the one or more tethers extends proximally through the lumen along an outer surface of the inner shaft, wherein the one of the one or more tethers is configured to be actuated by a user.

14. The stent delivery system of claim 13, wherein actuation of the one of the one or more tethers exerts a proximal force on the distal end region of the stent, causing the first end region to expand radially outward.

15. The stent delivery system of claim 12, wherein when the stent is in the second, deployed position, the inner shaft is proximally retracted and the one or more tethers disengage from the first end region of the stent and retract proximally with the inner shaft.

16. A method of treating a body lumen, the method comprising:
advancing a stent delivery system to a target tissue site, the stent delivery system including:
an outer shaft having a distal end region and an inner surface defining a lumen extending therein;
an inner shaft extending at least partially within the lumen of the outer shaft, the inner shaft having a stent receiving region disposed along a distal end region thereof;
a distal tip coupled to a distal end of the distal end region of the inner shaft, the distal tip having a plurality of hooks positioned adjacent a proximal edge of the distal tip;
a stent disposed along the stent receiving region, the stent having a first end region, a second end region, and a medial region positioned between the first end region and the second end region; and
one or more tethers coupled to the first end region of the stent, the one or more tethers configured to extend from the first end region to the distal tip and configured to engage with the plurality of hooks, thereby coupling the stent and the distal tip;
retracting the outer shaft proximally relative to the inner shaft to expose the first end region of the stent and permit the first end region of the stent to move from a first, pre-deployed position to a second, deployed position;
actuating the one or more tethers in a proximal direction, thereby applying a proximal force to the first end region of the stent causing the first end region to expand radially outward;
further retracting the outer shaft proximally relative to the inner shaft to expose the second end region of the stent and permit the second end region of the stent to move from the first, pre-deployed position to the second, deployed position; and
retracting the inner shaft proximally thereby causing the one or more tethers to disengage from the first end region of the stent and retract proximally with the inner shaft.

17. The method of claim 16 further comprising, while applying the proximal force to the first end region, positioning the first end region of the stent adjacent a first tissue structure such that the first tissue structure is moved in a proximal direction and engages with a second tissue structure.

18. The method of claim 16, wherein the one or more tethers includes a singular tether configured to be threaded within the first end region of the stent and form a plurality of loops configured to engage with the plurality of hooks.

19. The method of claim 16, wherein the first end region includes a first flange structure and the second end region includes a second flange structure, wherein the proximal force applied to the first end region causes the first flange structure to include an outer diameter greater than an outer diameter of the second flange structure.

20. The method of claim 16, wherein one of the one or more tethers extends proximally through the lumen along an outer surface of the inner shaft, wherein the one of the one or more tethers is configured to be actuated by a user.
